(19) United States
(12) Patent Application Publication
(10) Pub. No.: US 2009/0060783 A1
(43) Pub. Date: Mar. 5, 2009

(54) POLYMER CONCENTRATION MONITORING SYSTEM AND USE THEREOF

(76) Inventors: Kenneth Charles Barrett, Bristol, PA (US); Caibin Xiao, Harleysville, PA (US)

Correspondence Address: General Electric Company;GE Global Patent Operation, PO Box 861, 2 Corporate Drive, Suite 648, Shelton, CT, 06484, US

(21) Appl. No.: 11/849,662

(22) Filed: Sep. 4, 2007

(51) Int. Cl. G01N 33/00

(52) U.S. Cl. 422/62

(57) ABSTRACT

A system has been found which monitors and measures the concentration of polymer dispersant in industrial water systems, such as open recirculating, cooling tower water. The system comprises a polymer concentration monitor which is an in-line analyzer, and which has the ability to measure more than one characteristic or component at a time. The polymer concentration monitor is comprised of a filter module and a detection module. The incoming water first passes through the filter module and then on to the detection module and then out of the system.

According to one embodiment, the detection unit comprises a sample preparation unit, pumps, an optical measurement unit, a temperature measurement and control unit and an electronics unit. The optical measurement unit is comprised of a single or multiple photo optical components. The presence of more than one photo optical component provides the ability to perform more than one measurement at one time.

### BACKGROUND OF THE INVENTION

1. Field of the Invention

The field of the invention relates generally to the detection of components in industrial water systems, such as cooling and boiler water systems. More particularly, it relates to a system that monitors and measures the concentration of dispersants in industrial water systems.

2. Description of Related Art

It is well known to use water in numerous and varied industrial processes, such as removing heat from process equipment and the generation of steam. However, in most industrial processes it is not prudent or possible to use untreated water as the presence of impurities may impact the process in question. For example, compounds that inhibit scale formation are added to cooling tower and boiling water to prevent the formation or disposition of scale on the equipment used in these processes.

Most industrial waters contain metal cations such as calcium, barium, magnesium and sodium, as well as anions such as bicarbonate, carbonate, sulfate, phosphate and fluoride. When combinations of the cations and anions are present above certain concentrations, reaction products precipitate on the surface of the equipment in contact with the water in the process and form scale or deposits. The presence of such scale or deposits results in non-optimum process conditions and results in the cleaning or removal of such scale or deposits that is expensive and burdensome in that it often requires a shutdown of the process or system. Accordingly, in order to prevent such scale or deposits from occurring, it is preferable to treat the water with the proper chemicals in order to prohibit their formation.

One such method of controlling the scale and preventing fouling of heat exchanger surfaces by dirt, rust and inorganic precipitants such as phosphates or sulfates, is the inclusion of polymer dispersants as key ingredients in chemical treatment programs set up to address the issue of fouling of water systems. However, for the polymer dispersants to be effective, they must be maintained at desired concentration in the water systems, such as cooling towers. Therefore, a method for monitoring the concentration of such dispersants and other components is desired.

U.S. Pat. No. 6,635,224 discloses an online monitor system for continuous determination of the status of polymer containing process stream through the measurement of polymer molecular weight and/or size and the measurement of composition and concentration of selected species such as monomers and endgroups. In U.S. Pat. No. 6,072,576 a method for an online control of a process plant is taught, which has a plurality of steps and controls various properties, including viscosity, molecular weight, and molecular weight distribution.

Other monitoring systems known include on-line determination of polymer properties in a continuous polymerization reactor, as taught in U.S. Pat. No. 5,065,336 and a system and method for the automatic sampling and dilution of homogeneous particle dispersions as disclosed in U.S. Pat. No. 5,907,108. Unfortunately, each of these processes provides for one function or characteristic to be monitored and/or measured at a time.

Accordingly, a need exists for a system that would provide a means to monitor more than one component or characteristic at a time.

### SUMMARY OF THE INVENTION

A system has been found which monitors and measures the concentration of polymer dispersant in industrial water systems, such as open recirculating, cooling tower water. The system comprises a polymer concentration monitor which is an in-line analyzer, and which has the ability to measure more than one characteristic or component at a time. The polymer concentration monitor is comprised of a filter module and a detection module. The incoming water first passes through the filter module and then on to the detection module and then out of the system.

According to one embodiment, the detection unit comprises a sample preparation unit, pumps, an optical measurement unit, a temperature measurement and control unit and an electronics unit. The optical measurement unit is comprised of a single or multiple photo optical components. The presence of more than one photo optical component provides the ability to perform more than one measurement at one time.

The various features of novelty which characterize the invention are pointed out with particularity in the claims annexed to and forming a part of this disclosure. For a better understanding of the invention, its operating advantages and benefits obtained by its uses, reference is made to the accompanying drawings and descriptive matter. The accompanying drawings are intended to show examples of the many forms of the invention. The drawings are not intended as showing the limits of all of the ways the invention can be made and used. Changes to and substitutions of the various components of the invention can of course be made. The invention resides as well in sub-combinations and sub-systems of the elements described, and in methods of using them.

### BRIEF DESCRIPTION OF THE DRAWINGS

Refer now to the figures, which are meant to be exemplary and not limiting, and wherein like elements are numbered alike, and not all numbers are repeated in every figure for clarity of the illustration.

FIG. 1 is an example of a typical cooling tower flow scheme comprising a polymer concentration module as described herein;

FIG. 2 is a process flow diagram illustrating the relationship between the components of the polymer concentration module according to one embodiment of the invention;

FIG. 3 illustrated polymer concentration calculated from the multivariate equation plotted against actual concentration.

### DETAILED DESCRIPTION OF THE INVENTION

The singular forms “a,” “an” and “the” include plural referents unless the context clearly dictates otherwise.

The modifier “about” used in connection with a quantity is inclusive of the stated value and has the meaning dictated by the context (e.g., includes the degree of error associated with measurement of the particular quantity).

“Optional” or “optionally” means that the subsequently described event or circumstance may or may not occur, or that the subsequently identified material may or may not be present, and that the description includes instances where the event or circumstance occurs or where the material is present, and instances where the event or circumstance does not occur or the material is not present.

A system has been found which monitors and measures the concentration of polymer dispersant in industrial water systems, such as open recirculating, cooling tower water. The system comprises a polymer concentration monitor which is an in-line analyzer. The polymer concentration monitor is comprised of a filter module and a detection module. The incoming water first passes through the filter module and then on to the detection module and then out of the system.

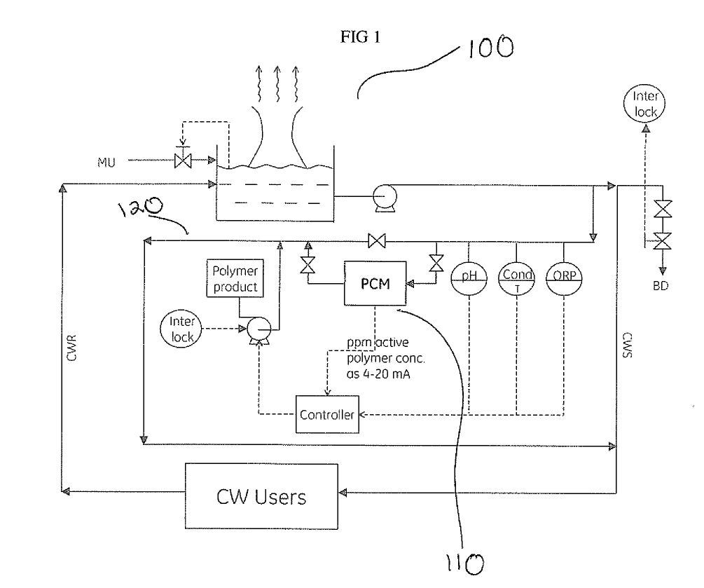

Referring to FIG. 1, a typical cooling tower flow scheme is shown 100, comprising a polymer concentration monitor (PCM) system 110. The monitor is shown installed in the chemical treatment loop where there may be other existing sensors, such as related to pH or conductivity monitoring. The PCM system as shown is connected to a controller. The PCM system may be installed anywhere within the system it is to monitor where it can tap into the water loop to be monitored. In a cooling tower system, for example, it should be tied into the cooling water loop 120, preferably the supply side of the loop. Additional considerations are the necessity of the PCM system to have access to AC power and where electrical area classification is not hazardous.

FIG. 2 illustrates a process flow diagram of an embodiment of the present invention. The relationship between the components within the PCM system are illustrated 205. The illustrative detection module is comprised of a sample preparation unit, three pumps, an optical measurement unit, a temperature measurement and control unit and a programmable electronics unit. As demonstrated in FIG. 2, which is illustrative of a cooling water process, the water is processed through the filter module which is comprised of a filter or a combination of filters 205. Thereafter the water passes through a sample preparation unit, which is comprised of a sample pump 215, a level-switch sample cup and solenoid valve 210. The sample pump 215 draws water from the sample cup 210 and delivers it to the mixing cross 220. Also provided to the mixing cross are two additional water solutions, one comprising a buffer reagent 225 and one comprising a dye reagent 230. The three streams are controlled by three pumps 235, such as micro positive-displacement pumps. After the three streams are combined in the mixing cross, the mixture flows to the optical measurement unit 240.

The optical measurement unit, also referred to as the flow cell, is comprised of optical components which detect the reaction color change 240. The optical measurement unit is comprised of a pair or pairs of LEDs (light-emitting diodes) and photodiodes (PD), an optical detection zone and a LED/PD holder. An additional component of the PCM system is the temperature measurement and control unit, which is comprised of a thermocouple 245, a heater 250 and a control unit. This temperature unit is installed in a location to measure either the ambient air temperature inside the PCM or immersed in the reaction mixture to measure the liquid temperature. The final unit to be included in the PCM system is the electronic unit, which is the brains of the PCM as it controls the functions of the other units, including but not limited to the heater 250, the pumps 235, the switches, and calculations of polymer concentration.

One embodiment of the invention provides for a filter module that comprises a pair of alternating coarse filters 205 that prevent large size particles from entering the water process and damaging the equipment. The coarse filters remove larger particulate matter from inlet water to prevent clogging of the pump and other fluidic components. The module is comprised of two mesh stainless steel filters and a four-way solenoid valve 255. The size of the mesh can vary depending on the water system involved, such as 500 mesh filters, to filter out particulates greater than or equal to about 50 microns. When the two-way valve 260 is cycled in its alternate position, one filter provides filtered water to the sample cup 210 while the other filter is being backwashed. After being subjected to the filter module the water proceeds to the detection module.

In one embodiment of the invention, the detection module is comprised of a sample preparation unit, pumps, an optical measurement unit, a temperature measurement and control unit and an electronics unit.

The sample preparation unit lets down the pressure of the inlet water from header pressure to atmospheric. By lowering the pressure, the sample pump 215 is protected, as the sample pump is rated for only about 5 psig. The sample preparation unit is comprised of a level-switch sample cup 210 and a solenoid valve. The level-switch sample cup is comprised of a pair of lead wires. When the cup is full, or at a designated high level, the two wires are electronically connected, which triggers the shutoff of the solenoid valve. When the cup is empty, or at a designated low level, the two wires are disconnected, which triggers the opening of the solenoid valve. The dead band between these two states is about 1.5 ml. The sample pump 215 draws water from the sample cup 210 and delivers it to the mixing cross 220.

Further comprised as part of the detection module are pumps. Three micro positive-displacement pumps maintain accurate volumetric delivery of three streams (sample, buffer, and dye) that are mixed together to produce the calorimetric reaction. Each pump delivers a constant stroke volume at a stroke frequency which is controlled by the programmable logic control (“PLC”) in the electronics unit, as described below 265. The buffer 225 and dye 230 are both reagents in dilute water solutions. They are mixed with the water sample in a mixing cross 220, then the mixture flows to the optical measurement unit. The resultant color change is proportional to the polymer concentration in the sample.

Another part of the detection unit is an optical measurement unit. The optical measurement unit is also frequently referred to as the flow cell 240. This unit comprises the optical components that detect the reaction color change. A unique aspect of the present invention is that it may have a single pair or multiple pairs of photo optical components. In one embodiment, the flow cell 240 comprises three pairs of photo optical components, each pair comprised of a light emitting diode (“LED”), and a photodiode (“PD”). The photo optical components further comprise an optical detection zone, and a LED/PD holder. An LED is a semiconductor device that emits incoherent narrow-spectrum light when electrically biased in the forward direction of the p-type-n-type semiconductor junction. This effect is a form of electroluminesence.

The optical path length is defined by the inner diameter of tubing, such as an ⅛″ OD tubing, which traverses the central through-hole of the LED/PD holder. No custom designed optical window is used. The OD tubing connects directly to the mixing cross 220 where the three streams, the water sample, the buffer and the dye streams, are mixed to produce the color change.

In one embodiment comprising three pairs of photo optical components, three LEDs and three photodiodes are installed in six radial channels perpendicular to the center through hole. The three LEDs generate incident light at different wavelengths, and the three corresponding photodiodes detect the respective transmittance on the opposite sides. The LEDs used include a tricolor with 467 nm (blue), 530 nm (green), and 634 nm (red) lights; an orange LED with 610 nm maximum and light green LED with 586 nm maximum emission. This configuration drastically simplifies the design and maintenance of the optical components. The three pairs of photo optical components provide the ability to measure three functions at a time. There is no maximum number of pairs of photo optical components that may be included, however the number will be affected by size limitations based on the intended use of the monitoring system.

The effluent from the optical measurement unit, comprising the mixed sample water and reagents, exits the polymer concentration unit and connects to a drain or a collection drum, depending on each plant's permitting requirements. Since the effluent is a non-hazardous wastewater, it is commonly discharged to a gravity drain.

The detection unit further comprises a temperature measurement and control unit. The function of this unit is to keep the temperature inside the polymer concentration monitor above a specified value, to prevent the water-based reagents from freezing and to minimize the temperature swings which have a significant effect on the calorimetric response. This unit is comprised of a thermocouple 245, a heater 250, and a control unit. The control action may be provided either by a programmable electronics unit or by a thermostat. A thermocouple 245 may be installed in a location to measure the ambient air temperature inside the monitor or immersed in the reaction mixture to measure the liquid temperature. The measured temperature is one of variables in the calibration equation.

As previously stated, an additional component of the detection module is the brains behind the polymer concentration monitor. This component is named the electronics unit, and may be referred to as the brain of the monitoring system in that it controls the functions of the other units, including, but not limited to, heaters 250, the switches for the filters 205, the filing of the sample cup 210, sequences and actuates the pumps and valves, receives signals from the optical measurements, and calculates polymer concentration for display. This electronics unit further comprises a one point calibration function that allows the user to adjust the reported concentration based on a standard with a known polymer concentration.

The actual process followed by the PCM to monitor the system is quite straightforward. The analyzer mixes the filtered sample with buffer and dye reagents to develop a calorimetric response to the polymer concentration. The polymer concentration is transmitted via analog signal to a chemical pump controller, such as the Pacesetter Platinum™, to control polymer concentration levels. The filtered sample is then routed to a sample cup 210. A level switch in the sample cup 210 causes a solenoid pinch valve upstream to open or close to keep the sample cup filled. A solenoid pump, connected downstream of the sample cup, constantly pumps small quantities of sample through the analyzer to minimize fouling of the tubing. Two reagents pumps are attached to the sample stream via a four-way cross. This configuration allows the reagents to be introduced to the sample stream and provides for sufficient mixing of the reagents with the sample to obtain a homogenous mixture. The mixture is moved through a detection zone where absorbance values corresponding to different LED lights are measured. The polymer concentration is calculated from the measured absorbance values.

The following steps constitute a full single measurement cycle:
1. The electronics unit sends an open signal to the sample valve to fill the sample cup with fresh sample.
2. The photodiode response is measured when each LED light is turned on in sequence. This response is referred as to the baseline value, denoted as R0, G0, B0, O0, and Lg0, respectively for the red, green, blue, orange, and light green LED lights.
3. The two reagent pumps are actuated five times. The buffer solution, dye solution, and sample are introduced to the mixing cross.
4. The electronics unit records the photodiode responses corresponding to the five LED lights as a function of time. As a result, five time profiles R(t), G(t), . . . , Lg(t) are produced during the course of the mixed fluidic packet passing through the detection zone.
5. The time profiles R(t), . . . , Lg(t) are analyzed by means of noise filtration algorithm. The minimum values Rmin, . . . , Lgmin in the profiles are obtained from the analysis.
6. Absorbance values AR, . . . , AG are calculated from the measured baseline values and the minimum values as AR=log(R0/Rmin), . . . , ALg=log(Lg0/Lg).
7. Polymer concentration is calculated from values AR, . . . , Alg based on a previously determined calibration equation.

### EXAMPLE

A comparative process was conducted to measure the absorbance values AR, AG, AO and ALg, obtained during a calibration run, using polymer standards at the known concentrations listed in the initial column. The results in Table 1 are the result of starting with clean tubing. The results of Table 2 were obtained after the tubing was heavily stained intentionally by shortening the flushing time between two consecutive dye injections. Although the calibration slope for each light (absorbance change perppm polymer) also listed in Table 1 is quite small, the measurement precision is still reasonable because the standard deviation of absorbance is also small. Since AR, AO and ALg are absorbance values measured by different lights with independent emission maxima, and they all increase with polymer concentration, the signal-go-noise ratio in the sum of AR+AO+ALg is usually greater than the individual absorbance values.

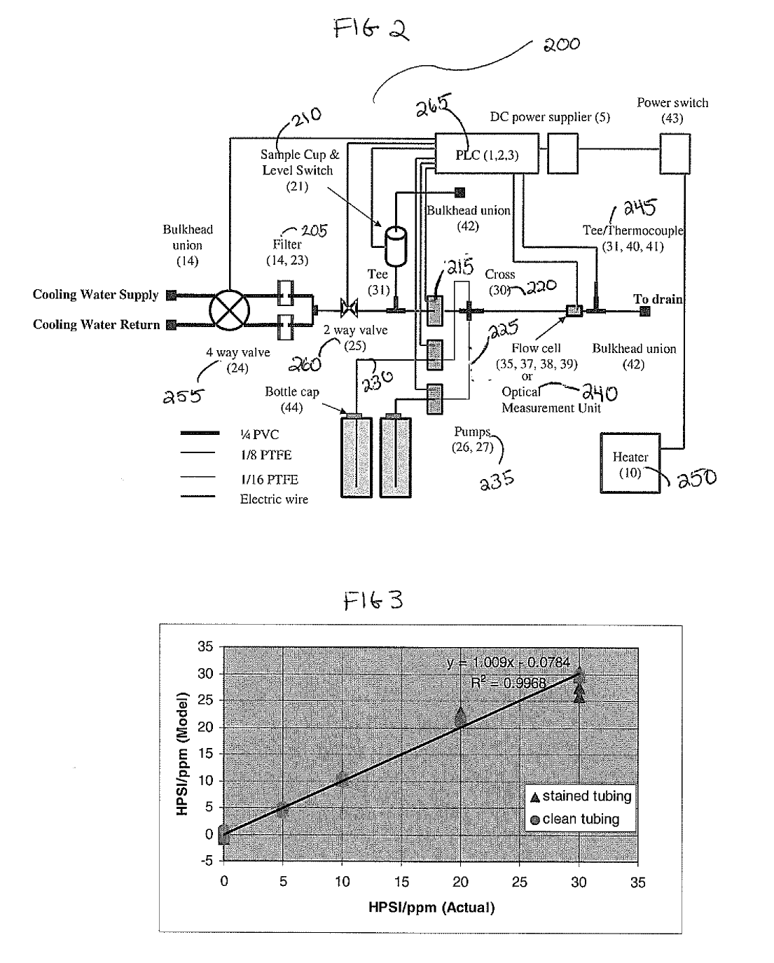

Although absorbance values measured from the clean tubing are higher than those from the stained tubing, a multivariate analysis in FIG. 3 shows that the two sets of data can be fitted into a single calibration equation:

Polymer/ppm=19.82+571.81R+281.90G−980.84O+50.00Lg−141.73R/G+136.13O/G

TABLE 1
Raw absorbance data obtained during a calibration run for polymer
standards with clean tubing

| Polymer/ppm | AR | Ag | Ao | Alg |
|---|---|---|---|---|
| 5 | 0.155434 | 0.193572 | 0.20841 | 0.244937 |
| 5 | 0.154334 | 0.194791 | 0.205514 | 0.244095 |
| 5 | 0.153028 | 0.194985 | 0.206657 | 0.246059 |
| 5 | 0.154548 | 0.193928 | 0.20696 | 0.242541 |
| Standard | 0.000994 | 0.000678 | 0.001192 | 0.001482 |
| deviation | | | | |
| 10 | 0.14313 | 0.191196 | 0.187794 | 0.223795 |
| 10 | 0.143164 | 0.19161 | 0.18722 | 0.223841 |
| 10 | 0.142475 | 0.185372 | 0.187029 | 0.221973 |
| 10 | 0.138287 | 0.189079 | 0.188042 | 0.220319 |
| 10 | 0.141979 | 0.189552 | 0.185695 | 0.216621 |
| 10 | 0.141807 | 0.188752 | 0.187411 | 0.217192 |
| Standard deviation | 0.001814 | 0.002225 | 0.000825 | 0.003166 |
| 0 | 0.161151 | 0.188977 | 0.219786 | 0.257588 |
| 0 | 0.161715 | 0.186648 | 0.218878 | 0.253785 |
| 0 | 0.161442 | 0.187583 | 0.218715 | 0.255148 |
| 0 | 0.158681 | 0.187717 | 0.217484 | 0.256018 |
| 0 | 0.160791 | 0.186648 | 0.217732 | 0.262993 |
| Standard deviation | 0.00121 | 0.00096 | 0.00093 | 0.003568 |
| 20 | 0.124277 | 0.198459 | 0.158929 | 0.197482 |
| 20 | 0.122213 | 0.200172 | 0.158819 | 0.195905 |
| 20 | 0.125435 | 0.198211 | 0.159067 | 0.19532 |
| Standard deviation | 0.001632 | 0.001068 | 0.000124 | 0.001118 |
| 30 | 0.10872 | 0.210822 | 0.141826 | 0.18002 |
| 30 | 0.107905 | 0.209768 | 0.142475 | 0.17858 |
| 30 | 0.107727 | 0.21163 | 0.142075 | 0.177087 |
| 30 | 0.110067 | 0.21152 | 0.143606 | 0.177581 |
| Standard deviation | 0.001066 | 0.000856 | 0.000787 | 0.001294 |
| 0 | 0.161647 | 0.180073 | 0.217732 | 0.252621 |
| 0 | 0.159673 | 0.179208 | 0.217323 | 0.247976 |
| 0 | 0.160474 | 0.178641 | 0.218801 | 0.254504 |
| 0 | 0.159894 | 0.178206 | 0.21798 | 0.252725 |
| 0 | 0.159852 | 0.179826 | 0.21798 | 0.254479 |
| 0 | 0.160321 | 0.180261 | 0.217894 | 0.253614 |
| Standard deviation | 0.000722 | 0.000826 | 0.000484 | 0.002432 |
| Slope | −0.0018 | 0.0009 | −0.0026 | 0.0026 |
| R2 | 0.992 | 0.830 | 0.986 | 0.971 |

TABLE 2
Raw absorbance data obtained during a calibration run with
standards and stained tubing

| Polymer/ppm | AR | Ag | Ao | Alg |
|---|---|---|---|---|
| 0 | 0.157817 | 0.14526 | 0.187871 | 0.238435 |
| 0 | 0.156964 | 0.14284 | 0.193402 | 0.238535 |
| 0 | 0.161374 | 0.143681 | 0.189599 | 0.235186 |
| 0 | 0.161039 | 0.145781 | 0.187928 | 0.242698 |
| 0 | 0.15875 | 0.147567 | 0.192881 | 0.240408 |
| 0 | 0.162705 | 0.150741 | 0.198002 | 0.24746 |
| Standard deviation | 0.002259 | 0.00286 | 0.003931 | 0.004234 |
| 5 | 0.149864 | 0.149091 | 0.17902 | 0.231357 |
| 5 | 0.148392 | 0.148117 | 0.174233 | 0.226096 |
| 5 | 0.149441 | 0.150215 | 0.181397 | 0.230238 |
| 5 | 0.148291 | 0.148291 | 0.177148 | 0.231781 |
| Standard deviation | 0.000777 | 0.000957 | 0.003027 | 0.002598 |
| 10 | 0.138039 | 0.154725 | 0.163609 | 0.215147 |
| 10 | 0.137884 | 0.157213 | 0.161263 | 0.217689 |
| 10 | 0.138815 | 0.157678 | 0.163247 | 0.217074 |
| 10 | 0.139328 | 0.158641 | 0.165719 | 0.218423 |
| Standard deviation | 0.000677 | 0.001669 | 0.001826 | 0.001404 |
| 20 | 0.119755 | 0.176899 | 0.137109 | 0.193154 |
| 20 | 0.121887 | 0.17933 | 0.138893 | 0.193681 |
| Standard deviation | 0.001508 | 0.001719 | 0.001261 | 0.000373 |
| 30 | 0.11053 | 0.188042 | 0.122213 | 0.179456 |
| 30 | 0.110138 | 0.187526 | 0.124029 | 0.18241 |
| 30 | 0.102621 | 0.187851 | 0.126182 | 0.180712 |
| 30 | 0.108205 | 0.184043 | 0.122874 | 0.178271 |
| 30 | 0.110227 | 0.186076 | 0.124194 | 0.184627 |
| 30 | 0.112543 | 0.180147 | 0.124442 | 0.182729 |
| 30 | 0.113274 | 0.176401 | 0.127512 | 0.184057 |
| Standard deviation | 0.00352 | 0.00447 | 0.00183 | 0.002361 |

While the present invention has been described with references to preferred embodiments, various changes or substitutions may be made on these embodiments by those ordinarily skilled in the art pertinent to the present invention with out departing from the technical scope of the present invention. Therefore, the technical scope of the present invention encompasses not only those embodiments described above, but all that fall within the scope of the appended claims.

What is claimed is:

1. An online polymer concentration monitoring system comprising:
a filter module; and
a detection module, wherein the detection module comprises a sample preparation unit, pumps, an optical measurement unit, a temperature measurement and control unit and an electronics unit.

2. The monitoring system of claim 1 wherein the optical measurement unit comprises one photo optical unit.

3. The monitoring system of claim 1 wherein the optical measurement unit comprises multiple photo optical units.

4. An online polymer concentration monitoring system comprising:
a filter module comprising a combination of filters;
a sample preparation unit comprising a sample pump, a level-switch sample cup, and a solenoid valve;
an optical measurement unit comprising at least one pair of light-emitting diodes and photodiodes, an optical detection zone and a light-emitting diode/photodiode holder;
a temperature measurement and control unit comprising a thermocouple, a heater and a control unit; and
an electronics unit.

5. The monitoring system of claim 4 wherein the electronics unit controls the functions of the other units and additionally calculates polymer concentration.

6. The monitoring system of claim 4 wherein the light-emitting diode/photodiode holder comprises tubing traversing a central through-hole in the holder.

7. The monitoring system of claim 6 wherein the tubing is ⅛″ tubing.

8. The monitoring system of claim 4 which further comprises pumps responsible to deliver three water based streams, one containing a water sample from the sample preparation unit, one containing buffer reagent and one containing dye reagent.

9. The monitoring system of claim 8 wherein the electronics unit comprises a programmable logic control that defines stroke frequency and volume for pumps that deliver the three water based streams.

10. An industrial water cooling tower system comprising the online polymer concentration monitor system of claim 4.

11. The water cooling tower system of claim 10 wherein the polymer concentration monitor system is installed in the chemical treatment loop.
